(19) United States
(12) Patent Application Publication
(10) Pub. No.: US 2014/0350300 A1
(43) Pub. Date: Nov. 27, 2014

(54) ALISKIREN HEMIFUMARATE, CRYSTAL FORM AND AMORPHOUS SOLID

(71) Applicants: Domenico Badone, Lugano (CH); Lau C.K., Lugano (CH); Yuan Jiandong, Lugano (CH)

(72) Inventors: Domenico Badone, Lugano (CH); Lau C.K., Lugano (CH); Yuan Jiandong, Lugano (CH)

(73) Assignee: DJADA PHARMACEUTICAL SA, Lugano (CH)

(21) Appl. No.: 14/131,615

(22) PCT Filed: Jul. 10, 2012
(86) PCT No.: PCT/EP2012/063492

§ 371 Date: Jul. 7, 2014

(30) Foreign Application Priority Data
Jul. 11, 2011 (IT) MI2011A001290

(51) Int. Cl. C07C 237/20; C07C 231/22

(52) U.S. Cl. CPC C07C 237/20; C07C 231/22; C07B 2200/13
USPC 564/157

(57) ABSTRACT

Aliskiren hemi fumarate has novel distinctive physico-chemical properties. An amorphous solid is also made of the same Aliskiren hemi fumarate. process for the prepares the forms of Aliskiren hemifumarate.

This invention refers to a novel crystal form of Aliskiren hemifumarate, having the novel distinctive physico-chemical properties described herein and also to a novel amorphous solid of the same Aliskiren hemifumarate. A further object of this invention is a process for the preparation of said forms of Aliskiren hemifumarate.

### STATE OF THE ART

Aliskiren is an anti-hypertension that acts on the renin-angiotensin-aldosterone system, inhibiting this. In particular, Aliskiren is a direct inhibitor of renin. Renin is the enzyme that, secreted by the juxtaglomerular apparatus of the kidneys following sympathetic stimulation caused by a drop in blood pressure, converts angiotensinogen (inactive protein produced by the liver and normally in circulation) into Angiotensin 1 which is then converted by the ACE (Angiotensin Converting Enzyme) into Angiotensin 2, an active protein with vascular constriction effects that stimulates the production of aldosterone.

The chemical name of Aliskiren of formula (A) is (2S,4S,5S,7S)-5-amino-N-(3-amino-2,2-dimethyl-3-oxypropyl)-4-hydroxy-7-[[4-methoxy-3-(3-methoxypropoxy)pheny]methyl]-8-methyl-2-propan-2 ilnonanamide.

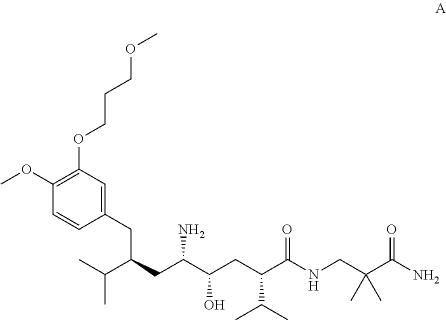 [SMILES: COCCCOc1cc(C[C@@H](C[C@H](N)[C@@H](O)C[C@H](C(=O)NCC(C)(C)C(N)=O)C(C)C)C(C)C)ccc1OC]

Known difficulties tied to the formulation of Aliskiren are to be ascribed principally to its highly hygroscopic nature and relatively low stability.

The hemifumarate salt of Aliskiren is known and used in therapy.

WO2009/064479 describes amorphous and polymorphic forms of Aliskiren hemifumarate and processes for their preparation. Crystal forms of Aliskiren hemifumarate are described in WO2008/061622. WO2009/143423 describes monofumarate Aliskiren and related preparation processes. The free base of Aliskiren is described in WO2009/149344.

The physical properties of a solid state active principle are fundamental in managing the material during transformation into a pharmaceutical product. Emphasis is laid, in particular, on flowability properties in addition to speed of dissolution in an aqueous liquid. The latter will influence the speed of dissolution of the active principle in the stomach of a patient, with evident therapeutic consequences. Considerable attention must be paid to dissolution speed also in the formulation of syrups, tonics and other liquid medicaments. These and other physical characteristics are influenced by the shape and orientation of the molecules in a unit cell which defines a particular polymorphic form of a substance. The polymorphic form may result in different thermal behaviour from that of the amorphous materials or another polymeric form. Thermal behaviour is measured in the laboratory with techniques such as the capillary melting point, thermogravimetric analysis (TGA) and differential scanning calorimetry (DSC) and may be used to distinguish certain polymorphic forms from others. A particular polymorphic form may also give rise to distinct spectroscopic properties, recordable through X-ray crystallography, NMR spectrometry and infrared spectrometry.

The discovery of novel forms of Aliskiren hemifumarate provides an opportunity to improve the process of synthesis of the active pharmaceutical ingredient (API), generating a form of Aliskiren hemifumarate with improved characteristics, for example, in terms of flowability and solubility, currently lacking in the state of the art.

A novel crystal form of Aliskiren hemifumarate, with distinctive, advantageous physico-chemical characteristics, is prepared, described and characterised here for the first time.

### DESCRIPTION OF THE INVENTION

The aim of this invention is to make available a novel crystal form and a novel amorphous solid of Aliskiren hemifumarate.

More complete understanding of this invention can be obtained referring to the summary tables of certain physico-chemical characteristics of Aliskiren hemifumarate provided below.

The main peaks of X-ray powder diffraction, the main bands and characteristic of the FT-IR spectrum, the thermogravimetric analysis are furnished.

The X-ray powder diffractogram (XRPD) has been obtained using the instrument X'Pert PRO PANalytical with single scan, using Kα1 radiation. The diffractogram is measured in reflection mode in the range 3-40° 2θ.

The FT-IR spectrum (Fourier transform IR spectroscopy) was recorded with the Nicolet FT-IR 6700 (ThermoFischer) appliance equipped with a KBr splitter and DTGS KBr detector. The spectrum was acquired in 16 scans at a resolution of 4 cm⁻¹ for the crystal form and the solvate, in 32 scans at a resolution of 4 cm⁻¹ for the amorphous solid.

DSC analyses were carried out using a differential scanning calorimeter DSC 200 F3 Maia®. The samples were loaded in an aluminium crucible and heated at 10° C./min in the temperature range from 20 to 450° C.

The thermograms were obtained using the STA 409 PC Luxx® Netzsch thermo-balance. The samples were loaded in an aluminium crucible and heated at 10° C./min in the temperature range from 25 to 490° C.

### DESCRIPTION OF THE FIGURES

FIG. 1: XRPD spectrum of Aliskiren hemifumarate, crystal form.

FIG. 2: FT-IR spectrum of Aliskiren hemifumarate, crystal form.

FIG. 3: DSC analysis of Aliskiren hemifumarate, crystal form.

FIG. 4: TGA analysis of Aliskiren hemifumarate, crystal form.

FIG. 5: XRPD spectrum of Aliskiren hemifumarate p-xylene solvate.

FIG. 6: FT-IR spectrum of Aliskiren hemifumarate p-xylene solvate.

FIG. 7: DSC analysis of Aliskiren hemifumarate p-xylene solvate.

FIG. 8: TGA analysis of Aliskiren hemifumarate p-xylene solvate.

FIG. 9: XRPD spectrum of Aliskiren hemifumarate, amorphous solid.

FIG. 10: FT-IR spectrum of Aliskiren hemifumarate, amorphous solid.

FIG. 11: DSC analysis of Aliskiren hemifumarate, amorphous solid.

FIG. 12: TGA analysis of Aliskiren hemifumarate, amorphous solid.

FIG. 13: dissolution speed of Aliskiren hemifumarate, amorphous solid (curve A) and Aliskiren hemifumarate, amorphous solid obtained as described in WO2008/061622 (curve B).

FIG. 14: XRPD spectrum of Aliskiren hemifumarate, amorphous solid obtained as described in WO2008/061622, before and after exposure for 5 days at 25° C., 60% RH.

FIG. 15: XRPD spectrum of Aliskiren hemifumarate, amorphous solid, before and after exposure for 5 days at 25° C., 60% RH.

### DETAILED DESCRIPTION OF THE INVENTION

The crystal form of Aliskiren hemifumarate claimed in this invention is preferably obtained through desolvation of Aliskiren hemifumarate solvate with p-xylene and is characterised by the following physico-chemical parameters.

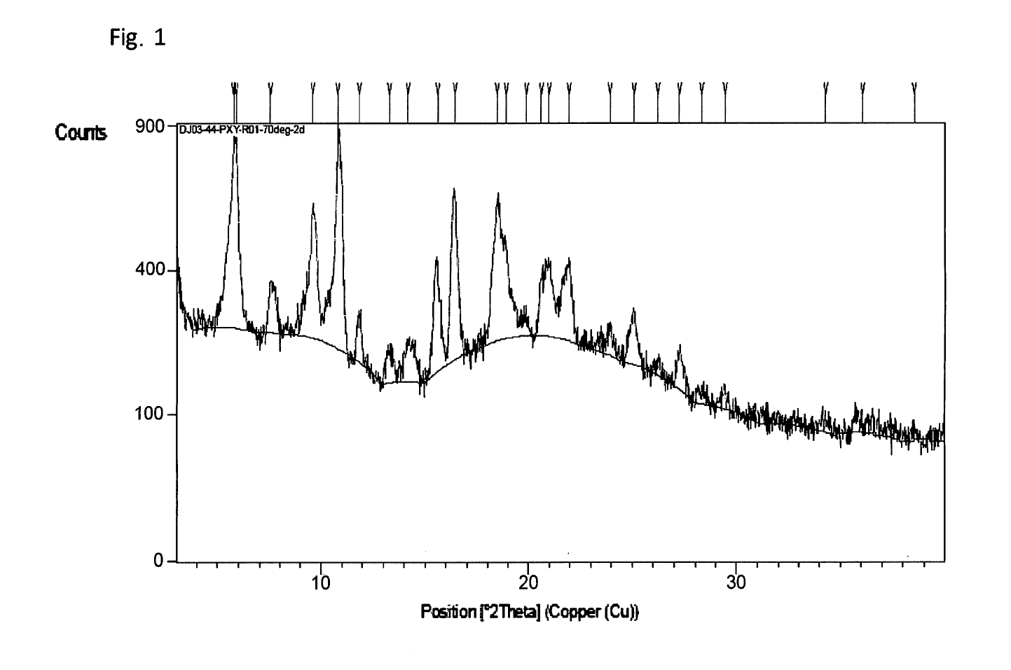

XRPD analysis makes it possible to obtain the characteristic spectrum shown in FIG. 1. Main peaks at 2theta+/−0.3 degrees are: 5.8, 5.9, 9.6, 10.8, 15.6, 16.4, 18.4, 18.9. Table 1 below shows the significant peaks of the spectrum.

TABLE 1

| | | FWHM | | |
|---|---|---|---|---|
| Pos. [° 2Th.] | Height [cts] | [° 2Th.] | d-spacing [Å] | Rel. Int. [%] |
| 5.7633 | 597.73 | 0.0836 | 15.33488 | 95.82 |
| 5.8783 | 597.72 | 0.1004 | 15.03525 | 95.82 |
| 7.5402 | 114.81 | 0.1673 | 11.72475 | 18.41 |
| 9.5848 | 356.92 | 0.3011 | 9.22775 | 57.22 |
| 10.8149 | 623.79 | 0.0836 | 8.18080 | 100.00 |
| 11.8491 | 103.35 | 0.2342 | 7.46899 | 16.57 |
| 13.3056 | 61.00 | 0.3346 | 6.65444 | 9.78 |
| 14.1782 | 76.24 | 0.2676 | 6.24684 | 12.22 |
| 15.6019 | 257.52 | 0.1673 | 5.67986 | 41.28 |
| 16.4443 | 429.99 | 0.1004 | 5.39074 | 68.93 |
| 18.4279 | 383.83 | 0.2007 | 4.81471 | 61.53 |
| 18.8711 | 229.28 | 0.1673 | 4.70262 | 36.76 |
| 19.8882 | 42.40 | 0.3346 | 4.46436 | 6.80 |
| 20.5874 | 132.29 | 0.1338 | 4.31429 | 21.21 |
| 20.9682 | 178.71 | 0.2676 | 4.23679 | 28.65 |
| 21.9324 | 188.48 | 0.4684 | 4.05266 | 30.22 |
| 23.9282 | 63.15 | 0.1673 | 3.71896 | 10.12 |
| 25.0750 | 114.37 | 0.1673 | 3.55142 | 18.34 |
| 26.2090 | 22.66 | 0.4015 | 3.40027 | 3.63 |
| 27.2420 | 67.56 | 0.2342 | 3.27364 | 10.83 |
| 28.3084 | 18.87 | 0.4015 | 3.15270 | 3.02 |
| 29.4345 | 24.70 | 0.2676 | 3.03459 | 3.96 |
| 34.2598 | 14.64 | 0.5353 | 2.61744 | 2.35 |
| 36.0523 | 15.38 | 0.8029 | 2.49131 | 2.47 |
| 38.5046 | 12.15 | 0.3346 | 2.33810 | 1.95 |

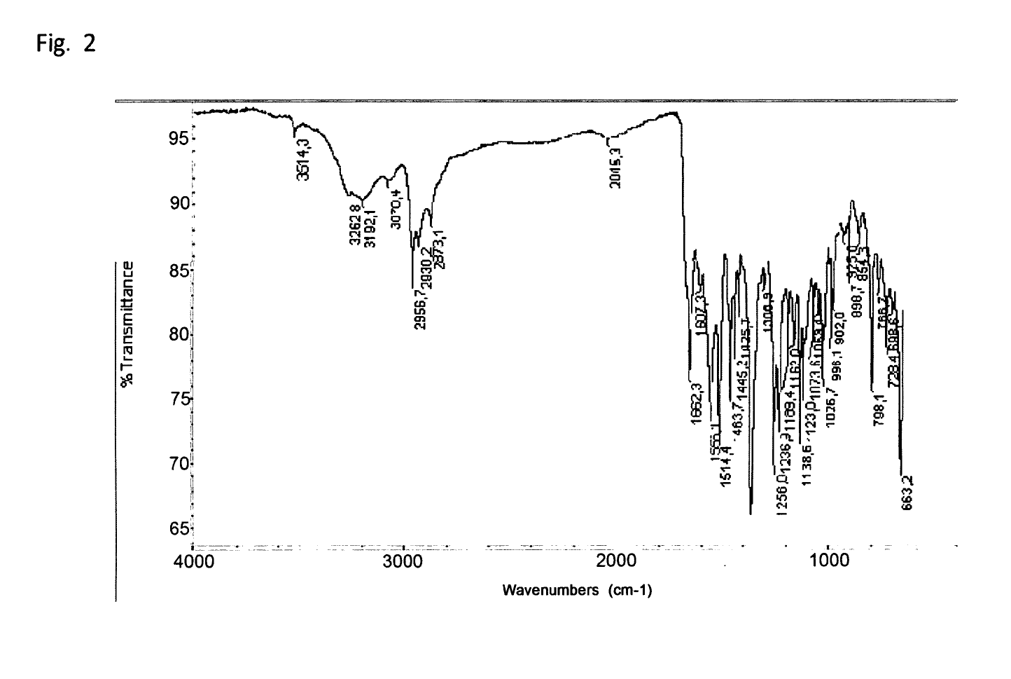

FT-IR analysis returns the spectrum shown in FIG. 2. Said FT-IR spectrum is characterised by the peaks shown in Table 2 below.

TABLE 2

| | |
|---|---|
| Position: 663.9 | Intensity: 68.908 |
| Position: 698.0 | Intensity: 91.597 |
| Position: 726.1 | Intensity: 81.065 |
| Position: 762.6 | Intensity: 81.773 |
| Position: 775.0 | Intensity: 82.082 |
| Position: 797.3 | Intensity: 69.092 |
| Position: 853.9 | Intensity: 85.605 |
| Position: 000.2 | Intensity: 00.946 |
| Position: 898.4 | Intensity: 86.998 |
| Position: 916.7 | Intensity: 87.173 |
| Position: 978.3 | Intensity: 80.897 |
| Position: 992.9 | Intensity: 84.683 |
| Position: 1026.2 | Intensity: 75.777 |
| Position: 1056.1 | Intensity: 80.286 |
| Position: 1070.1 | Intensity: 81.673 |
| Position: 1122.0 | Intensity: 70.931 |
| Position: 1136.7 | Intensity: 74.835 |
| Position: 1162.7 | Intensity: 78.027 |
| Position: 1196.9 | Intensity: 81.770 |
| Position: 1194.4 | Intensity: 81.956 |
| Position: 1240.1 | Intensity: 75.203 |
| Position: 1255.9 | Intensity: 71.745 |
| Position: 1307.1 | Intensity: 83.550 |
| Position: 1325.0 | Intensity: 83.911 |
| Position: 1365.1 | Intensity: 65.044 |
| Position: 1406.1 | Intensity: 84.236 |
| Position: 1426.2 | Intensity: 83.699 |
| Position: 1115.5 | Intensity: 91.206 |
| Position: 1406.2 | Intensity: 79.404 |
| Position: 1514.8 | Intensity: 69.827 |
| Position: 1557.4 | Intensity: 69.604 |
| Position: 1562.2 | Intensity: 71.282 |
| Position: 1506.7 | Intensity: 84.843 |
| Position: 1660.3 | Intensity: 78.785 |
| Position: 2051.1 | Intensity: 94.308 |
| Position: 2351.3 | Intensity: 93.825 |
| Position: 2872.2 | Intensity: 88.379 |
| Position: 2926.7 | Intensity: 88.276 |
| Position: 2959.4 | Intensity: 86.073 |
| Position: 3076.1 | Intensity: 91.143 |
| Position: 3195.0 | Intensity: 89.724 |
| Position: 3512.4 | Intensity: 94.192 |

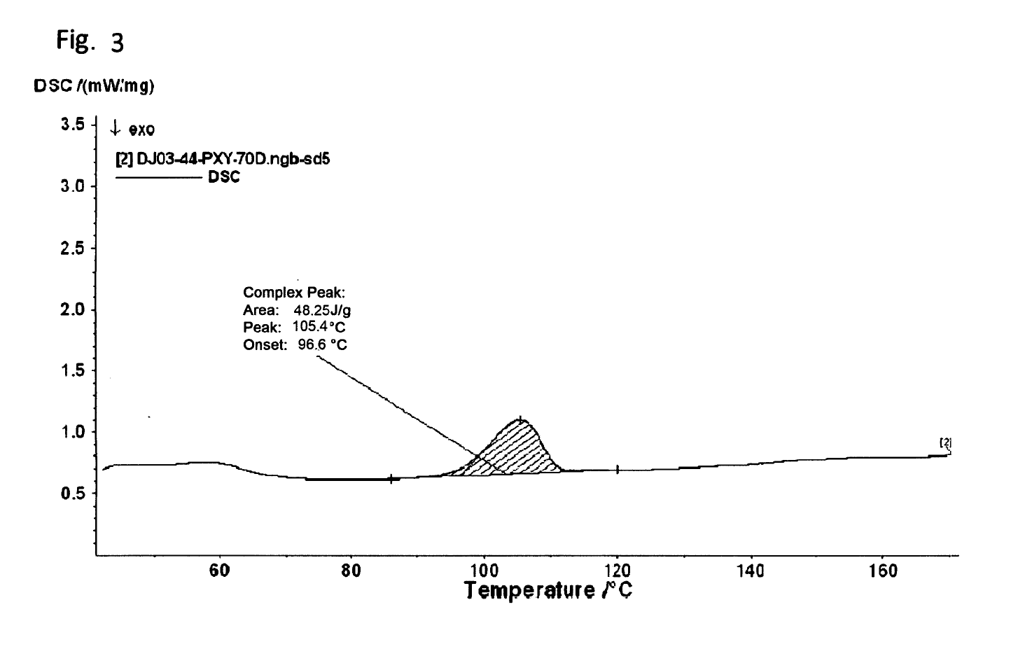

DSC analysis, shown in FIG. 3, highlights an endothermic peak, corresponding to the melting point at about 105.4° C., between 96.6 and 110° C.

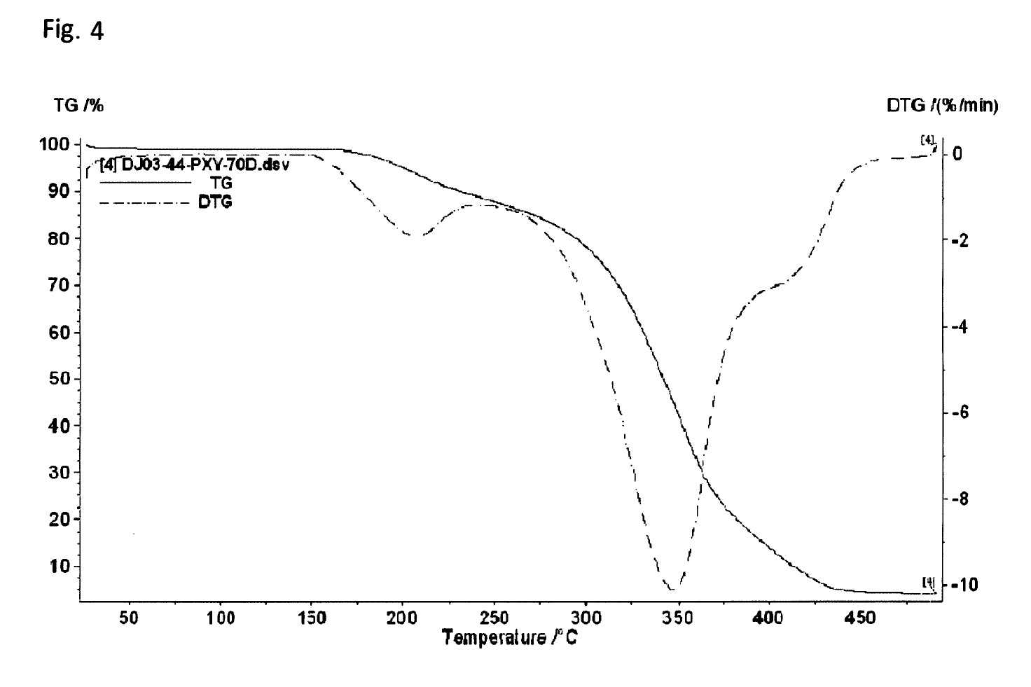

The thermogram shown in FIG. 4 highlights a continuous loss of weight on moving from about 150 to about 450° C. The characteristic events of the weight loss measured can be observed more clearly on the DTG curve, shown in the same plot. The DTG curve represents the derivative of the thermogram and makes it possible to observe events after 150° C., associated with downgrading of the sample following heating.

Said crystal form claimed here is obtained from Aliskiren hemifumarate p-xylene solvate. Said solvate form is characterised as follows.

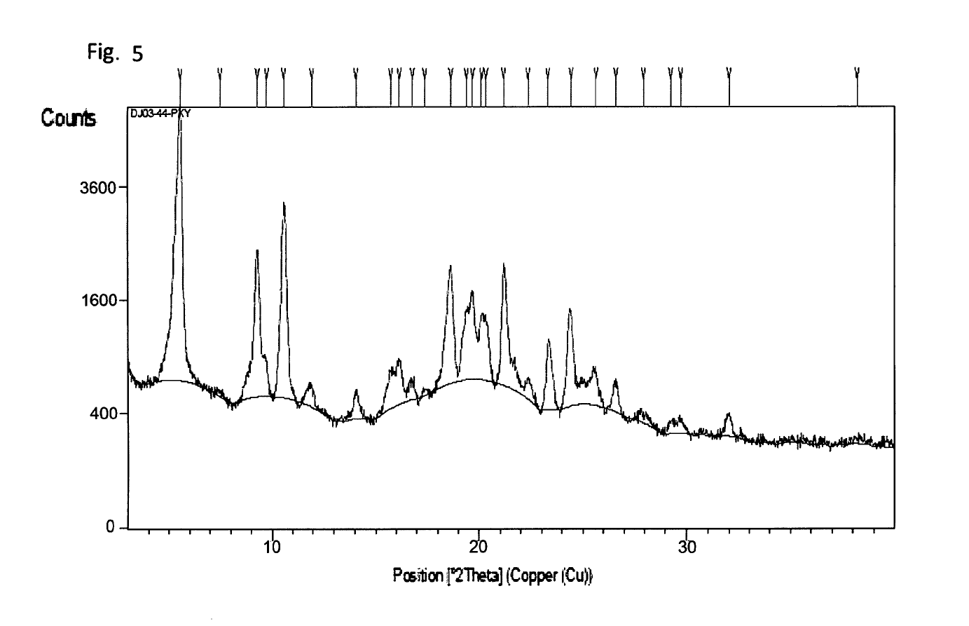

XRPD analysis of Aliskiren hemifumarate p-xylene solvate makes it possible to obtain the characteristic spectrum shown in FIG. 5. The main peaks at 2theta+/−0.3 degrees are: 5.5, 9.2, 10.5, 18.6, 19.7, 21.2. Table 3 below shows the significant peaks of the spectrum.

TABLE 3

| Pos. | Height | FWHM | d-spacing | Rel. Int. |
|---|---|---|---|---|
| [° 2Th.] | [cts] | [° 2Th.] | [Å] | [%] |
| 5.5076 | 4808.51 | 0.1840 | 16.04621 | 100.00 |
| 7.4486 | 50.00 | 0.4015 | 11.86870 | 1.04 |
| 9.2575 | 1835.92 | 0.1506 | 9.55327 | 38.18 |
| 9.6953 | 337.50 | 0.1171 | 9.12279 | 7.02 |
| 10.5420 | 2724.92 | 0.2342 | 8.39194 | 56.67 |
| 11.8937 | 168.37 | 0.4015 | 7.44108 | 3.50 |
| 14.0381 | 202.15 | 0.2007 | 6.30883 | 4.20 |
| 15.7150 | 329.28 | 0.1673 | 5.63921 | 6.85 |
| 16.1522 | 407.63 | 0.1171 | 5.48755 | 8.48 |
| 16.7616 | 155.18 | 0.2007 | 5.28939 | 3.23 |
| 17.3542 | 46.30 | 0.2007 | 5.11008 | 0.96 |
| 18.6016 | 1481.76 | 0.1673 | 4.77013 | 30.82 |
| 19.3592 | 775.22 | 0.1338 | 4.58512 | 16.12 |
| 19.6748 | 1024.19 | 0.1004 | 4.51230 | 21.30 |
| 20.1082 | 687.85 | 0.1338 | 4.41601 | 14.30 |
| 20.3377 | 659.96 | 0.2007 | 4.36668 | 13.72 |
| 21.1619 | 1389.92 | 0.1506 | 4.19843 | 28.91 |
| 22.3435 | 183.72 | 0.1673 | 3.97901 | 3.82 |
| 23.2894 | 594.83 | 0.1004 | 3.81950 | 12.37 |
| 24.4183 | 986.58 | 0.2007 | 3.64543 | 20.52 |
| 25.6492 | 271.69 | 0.2342 | 3.47320 | 5.65 |
| 26.5765 | 241.45 | 0.3011 | 3.35409 | 5.02 |
| 27.9078 | 65.55 | 0.5353 | 3.19703 | 1.36 |
| 29.2302 | 72.60 | 0.2007 | 3.05534 | 1.51 |
| 29.7079 | 89.32 | 0.2007 | 3.00729 | 1.86 |
| 32.0083 | 146.57 | 0.2676 | 2.79622 | 3.05 |
| 38.1913 | 30.53 | 0.5353 | 2.35655 | 0.63 |

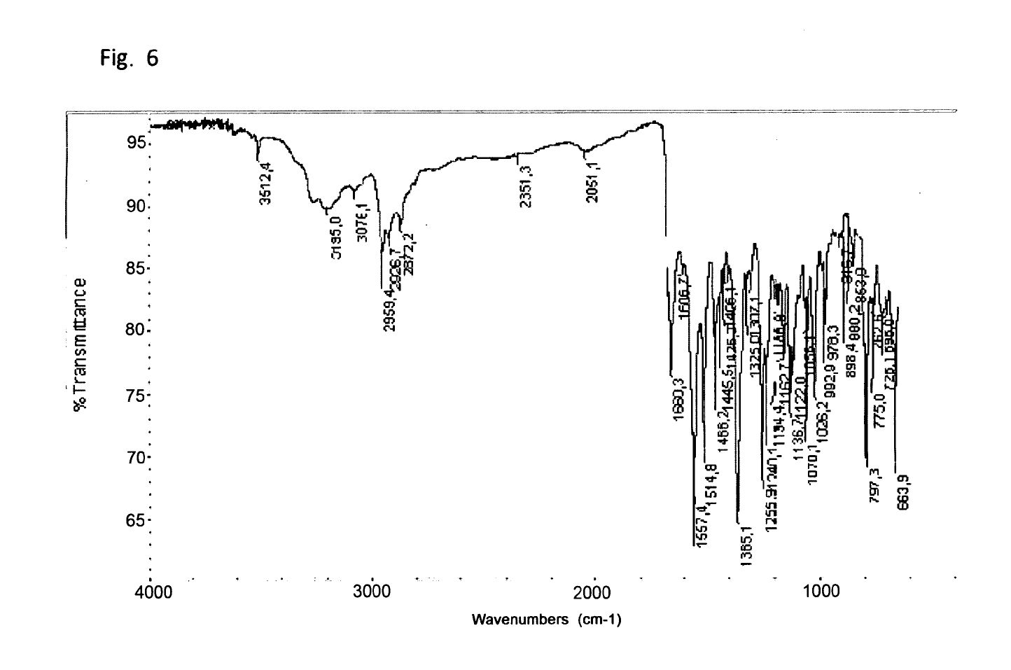

FT-IR analysis gives the spectrum shown in FIG. 6. Said FT-IR spectrum is characterised by the peaks show in Table 4 below.

TABLE 4

| | |
|---|---|
| Position: 663.2 | Intensity: 69.543 |
| Position: 698.6 | Intensity: 81.745 |
| Position: 728.4 | Intensity: 81.812 |
| Position: 776.7 | Intensity: 83.333 |
| Position: 798.1 | Intensity: 75.987 |
| Position: 854.3 | Intensity: 87.307 |
| Position: 898.7 | Intensity: 88.710 |
| Position: 925.0 | Intensity: 87.487 |
| Position: 982.0 | Intensity: 82.269 |
| Position: 996.1 | Intensity: 85.292 |
| Position: 1026.7 | Intensity: 79.391 |
| Position: 1053.1 | Intensity: 81.820 |
| Position: 1073.6 | Intensity: 82.624 |
| Position: 1123.0 | Intensity: 78.097 |
| Position: 1138.6 | Intensity: 77.012 |
| Position: 1162.0 | Intensity: 79.417 |
| Position: 1109.4 | Intensity: 01.046 |
| Position: 1236.9 | Intensity: 73.921 |
| Position: 1256.0 | Intensity: 73.153 |
| Position: 1906.9 | Intensity: 89.685 |
| Position: 1365.6 | Intensity: 66.447 |
| Position: 1425.7 | Intensity: 84.092 |
| Position: 1445.2 | Intensity: 81.642 |
| Position: 1463.7 | Intensity: 80.042 |
| Position: 1514.4 | Intensity: 71.805 |
| Position: 1156.1 | Intensity: 79.822 |
| Position: 1607.3 | Intensity: 83.473 |
| Position: 1662.3 | Intensity: 76.725 |
| Position: 2045.3 | Intensity: 94.916 |
| Position: 2873.1 | Intensity: 88.712 |
| Position: 2930.2 | Intensity: 87.270 |
| Position: 2956.7 | Intensity: 86.264 |
| Position: 3070.4 | Intensity: 91.736 |
| Position: 3192.2 | Intensity: 90.300 |
| Position: 3262.8 | Intensity: 90.609 |
| Position: 3514.0 | Intensity: 95.576 |

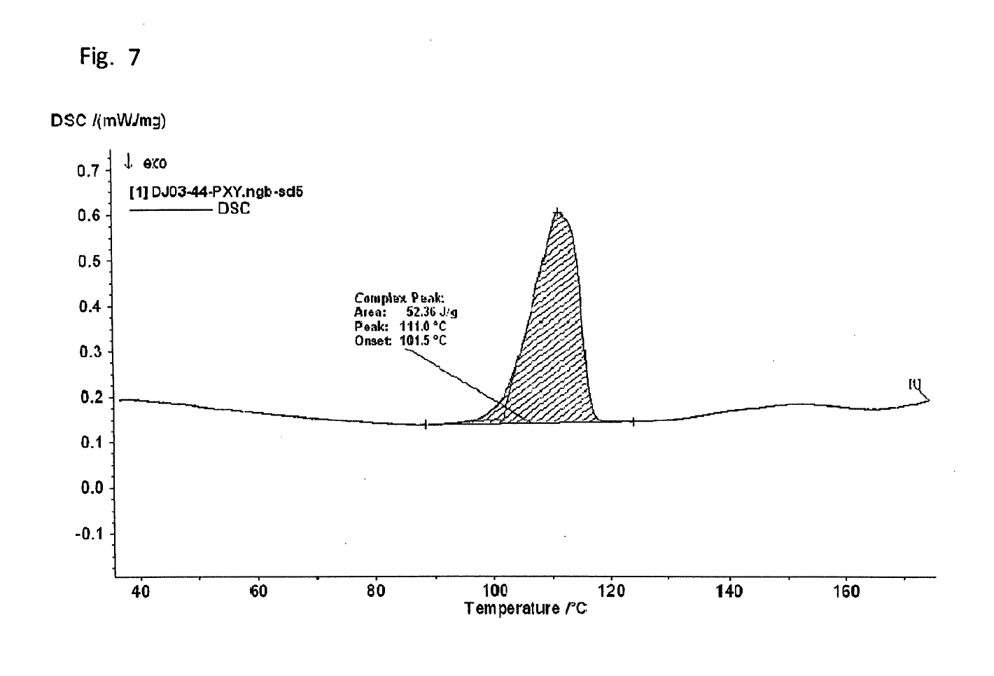

DSC analysis, shown in FIG. 7, shows an endothermic peak, corresponding to the melting point at about 111.0° C., between 101.5 and 118° C.

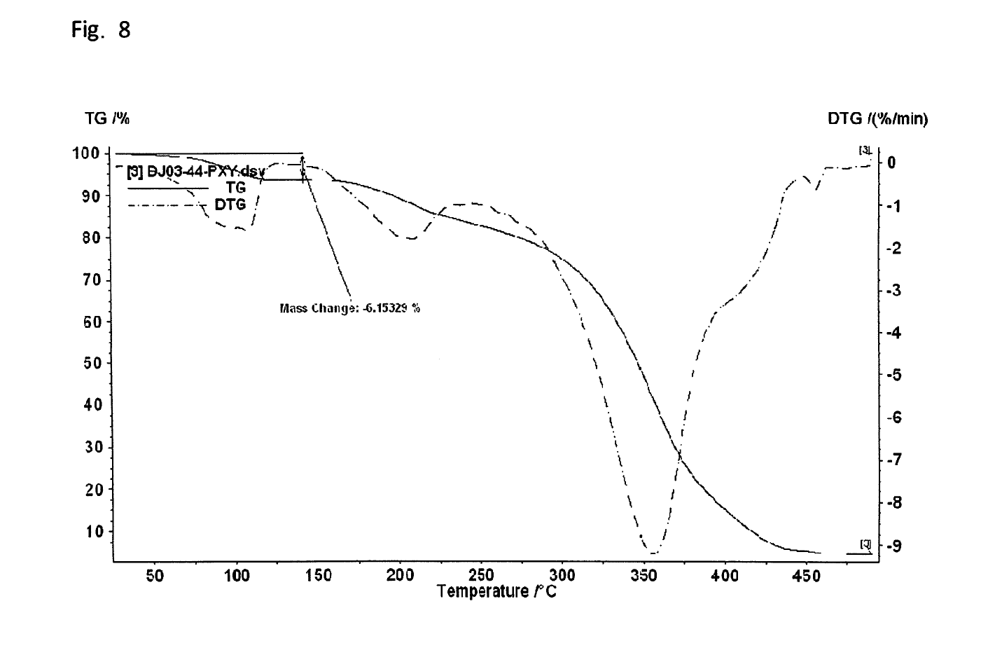

The thermogram shown in FIG. 8 highlights a continuous loss of weight, on moving from about 140 to about 450° C. The characteristic events of the weight loss measured can be observed more clearly on the DTG curve, shown in the same plot. The DTG curve makes it possible to observe a significant event with loss of weight of about 6.15% between 50° C. and 140° C. which corresponds to the loss of solvent following melting of the sample. The other events observed after 150° C. are associated with downgrading of the sample following heating.

Another object of this invention is Aliskiren hemifumarate, amorphous solid.

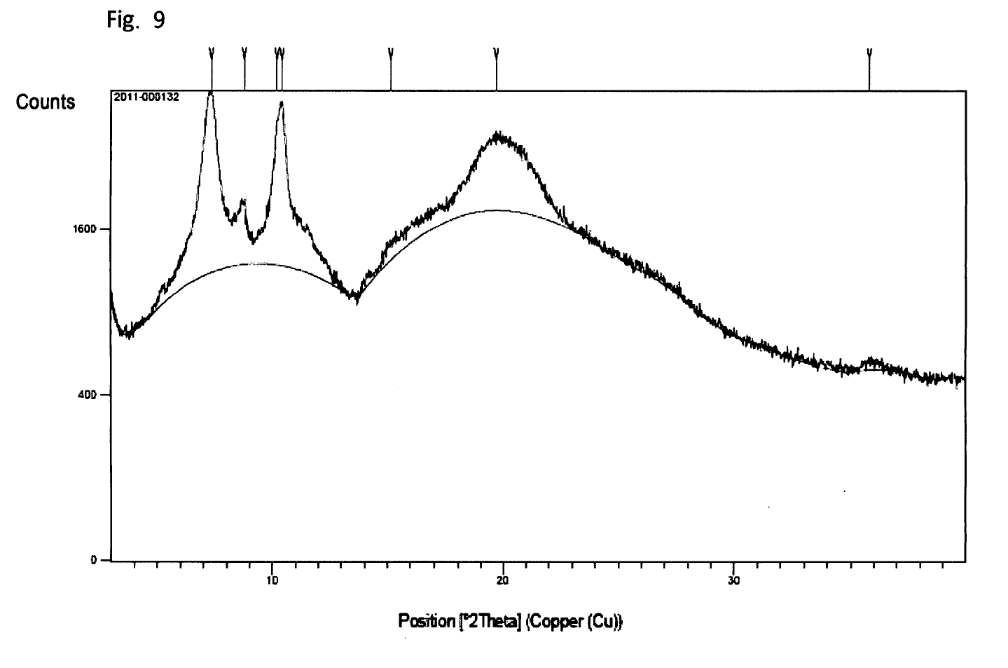

XRPD analysis of said Aliskiren hemifumarate, amorphous solid makes it possible to obtain the characteristic spectrum shown in FIG. 9. The main peaks at 2theta+/−0.3 degrees are: 7.3, 10.2, 10.4, 19.6. Table 5 below shows the significant peaks of the spectrum.

TABLE 5

| | | | | Rel. Int. |
|---|---|---|---|---|
| Pos. [° 2Th.] | Height [cts] | FWHM [° 2Th.] | d-spacing [Å] | [%] |
| 7.3453 | 1976.67 | 0.4349 | 12.03533 | 100.00 |
| 8.7655 | 577.68 | 0.2342 | 10.08828 | 29.23 |
| 10.1773 | 1530.19 | 0.1338 | 8.69183 | 77.41 |
| 10.3839 | 1796.14 | 0.2007 | 8.51933 | 90.87 |
| 15.0934 | 138.76 | 0.5353 | 5.87003 | 7.02 |
| 19.6590 | 816.31 | 0.5353 | 4.51588 | 41.30 |
| 35.7778 | 49.34 | 0.8029 | 2.50979 | 2.50 |

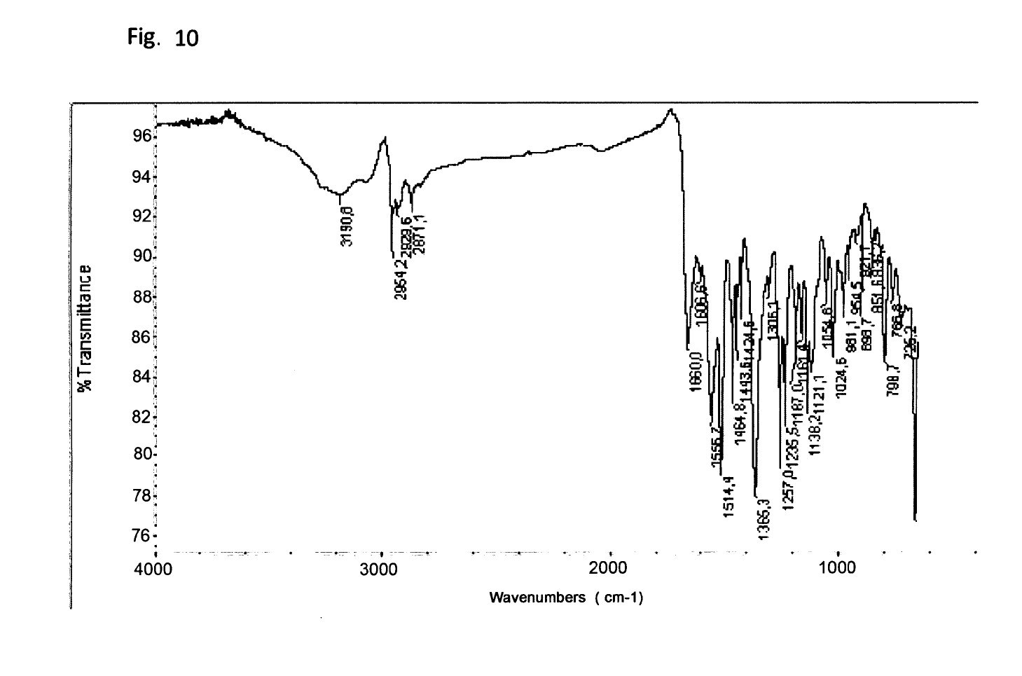

FT-IR analysis returns the spectrum shown in FIG. 10. Said FT-IR spectrum is characterised by the peaks shown in Table 6 below.

TABLE 6

| | |
|---|---|
| Position: 665.0 | Intensity: 76.922 |
| Position: 725.2 | Intensity: 86.850 |
| Position: 766.8 | Intensity: 87.945 |
| Position: 798.7 | Intensity: 84.949 |
| Position: 836.2 | Intensity: 91.106 |
| Position: 851.6 | Intensity: 90.731 |
| Position: 898.7 | Intensity: 91.806 |
| Position: 921.7 | Intensity: 90.972 |
| Position: 954.5 | Intensity: 90.325 |
| Position: 981.1 | Intensity: 87.554 |
| Position: 1024.6 | Intensity: 85.867 |
| Position: 1054.8 | Intensity: 87.804 |
| Position: 1121.1 | Intensity: 84.478 |
| Position: 1138.2 | Intensity: 84.439 |
| Position: 1161.4 | Intensity: 86.005 |
| Position: 1187.0 | Intensity: 87.166 |
| Position: 1235.5 | Intensity: 83.236 |
| Position: 1257.0 | Intensity: 82.138 |
| Position: 1306.1 | Intensity: 88.174 |
| Position: 1365.3 | Intensity: 78.142 |
| Position: 1424.6 | Intensity: 89.294 |
| Position: 1443.6 | Intensity: 87.930 |
| Position: 1464.8 | Intensity: 86.579 |
| Position: 1514.4 | Intensity: 79.276 |
| Position: 1556.7 | Intensity: 81.856 |
| Position: 1606.6 | Intensity: 88.973 |
| Position: 1660.0 | Intensity: 85.601 |
| Position: 2871.1 | Intensity: 92.567 |
| Position: 2929.6 | Intensity: 92.420 |
| Position: 2954.2 | Intensity: 92.135 |
| Position: 3190.8 | Intensity: 92.973 |

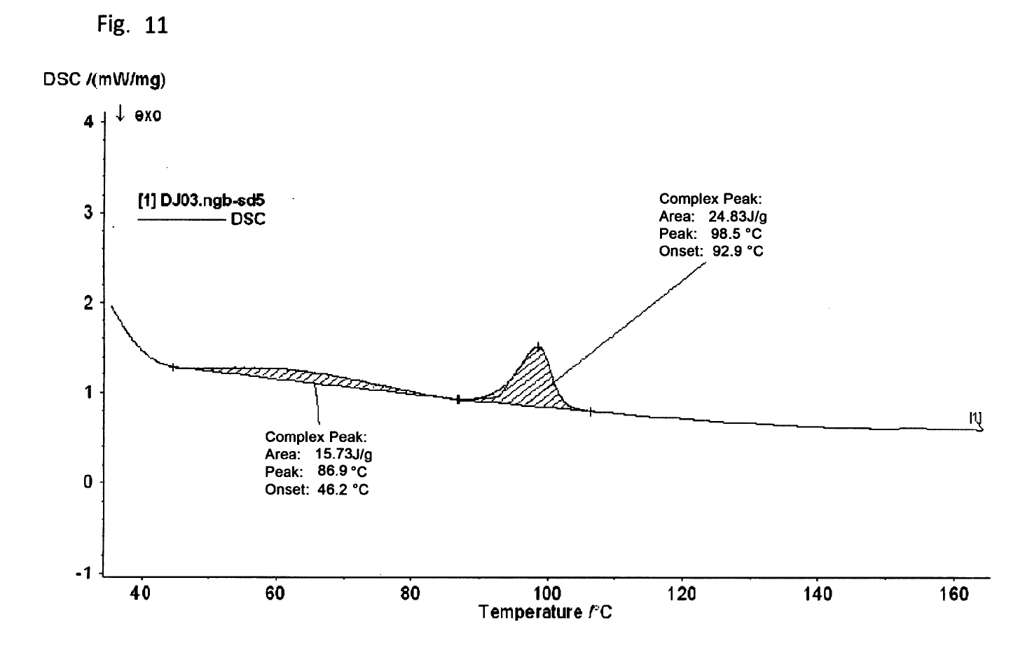

DSC analysis, shown in FIG. 11, highlights an endothermic peak, corresponding to the melting point at 98.5° C., between 87 and 07° C.

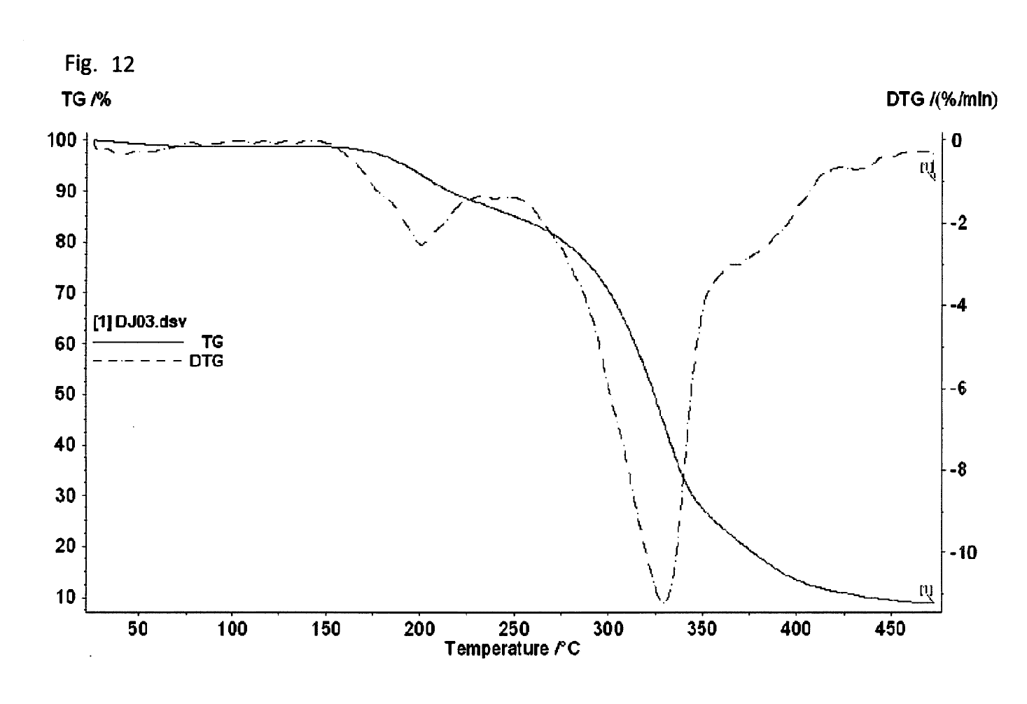

The thermogram shown in FIG. 12 shows a continuous loss of weight starting from about 170° C., loss of weight associated with decomposition of the sample after melting.

Another object of this invention is the process for the preparation of said crystal form of Aliskiren hemifumarate. In particular, said process comprises:
i) re-suspending Aliskiren hemifumarate in a suitable solvent, continuously stirred at room temperature in an oil bath;
ii) heating to a suitable temperature until a clear solution is obtained;
iii) cooling, continuing to stir the solution obtained in ii), to room temperature;
iv) stirring of the solution obtained in iii) at a suitable temperature and for a suitable time;
v) filtering of the mix obtained in iv) in order to isolate the precipitate;
vi) drying of the precipitate at a suitable temperature.

In a preferred embodiment, said phase ii) is carried out at a temperature of between 60 and 90° C., preferably at about 75° C. and said solvent is selected in the group that comprises benzene, toluene, xylene, preferably p-xylene. In said phase iii) said cooling is carried out slowly, preferably said solution reaches room temperature in about 5 hours, in oil bath. In said phase iv), said stirring is continued for a further 10 hours or more, preferably for about 12 hours, preferably at room temperature. In said phase vi), said drying takes place at a temperature of between 50 and 90° C., preferably at about 70° C., for about 3 hours.

The product obtained is the crystal form of Aliskiren hemifumarate claimed in this invention.

A further object of this invention is the process for the preparation of said amorphous solid of Aliskiren hemifumarate. In particular, said process comprises:
i) dissolving Aliskiren hemifumarate in alcohol and heating to a suitable temperature;
ii) cooling the solution obtained in i) to room temperature and spiking of the same with pure Aliskiren hemifumarate;
iii) stirring the mix obtained in ii) at a suitable temperature and for a suitable time;
iv) further cooling the mix and continuation of stirring;
v) filtering the mix obtained in iv) so as to isolate the precipitate;
vi) washing the precipitate with alcohol and drying under vacuum at a suitable temperature.

In another preferred embodiment, said phase i) is carried out at a temperature of between 35 and 55° C., more preferably at about 40-45° C., and said alcohol is selected in the group that comprises methanol, ethanol, propanol, butanol, isopropanol, isobutanol, preferably isopropanol. In said phase iii), stirring is continued for 10-20 hours, preferably for about 15 hours, at a temperature of between about 20 and about 25° C. In said phase iv), cooling is carried out preferably at a temperature below 10° C., preferably at a T of between about 0 and about 5° C. and said stirring is continued for a further 2 hours or more. In said phase vi), the alcohol is selected in the group that comprises methanol, ethanol, propanol, butanol, isopropanol, isobutanol, preferably cold isopropanol

Preferably, said washing is repeated twice. Drying under vacuum is carried out at a temperature below 50° C., preferably below about 40° C.

The product obtained is the amorphous form of Aliskiren hemifumarate claimed in this invention.

Said crystal form and said amorphous solid of Aliskiren hemifumarate can be applied in pharmaceutical compositions. The pharmaceutical composition that comprises said crystal form and/or said amorphous solid may contain additives such as sweeteners, aromas, coating substances, inert diluents such as lactose and talcum, binders such as starch, hydroxyethylcellulose, hydroxypropylcellulose and similar. Any conventional technique can be used for preparation of pharmaceutical formulations in accordance with this invention.

### EXAMPLE 1

### Preparation of Aliskiren Hemifumarate, Crystal Form

500 g of Aliskiren hemifumarate were re-suspended in 4 ml of p-xylene, stirring at room temperature in oil bath. The suspension was heated to 75° C. to obtain a clear solution. The solution was then placed in an oil bath in order to bring this, over a period of 5 hours, to room temperature while continuing stirring. Stirring continued for about 12 hours. After filtration, the filtrate was dried at 70° C. for about 3 hours and analysed using XRPD. The product obtained is the crystal form of Aliskiren hemifumarate claimed in this invention.

### EXAMPLE 2

### Preparation of Aliskiren Hemifumarate, Amorphous Solid

100 g of Aliskiren hemifumarate were dissolved in 850 ml of isopropyl alcohol, stirring at 40-45° C. After cooling to room temperature, the mix was spiked with pure Aliskiren hemifumarate and stirred at 20-25° C. for 15 hours. The mix was then cooled to 0-5° C., continuing stirring for another 2 hours. After filtration, the filtrate was washed twice with isopropyl alcohol, using 200 ml of cold isopropyl alcohol for each washing. The washed product was dried under vacuum at 40° C. and analysed by means of XRPD. The product obtained is Aliskiren hemifumarate amorphous form claimed in this invention.

### EXAMPLE 3

### Dissolution Test

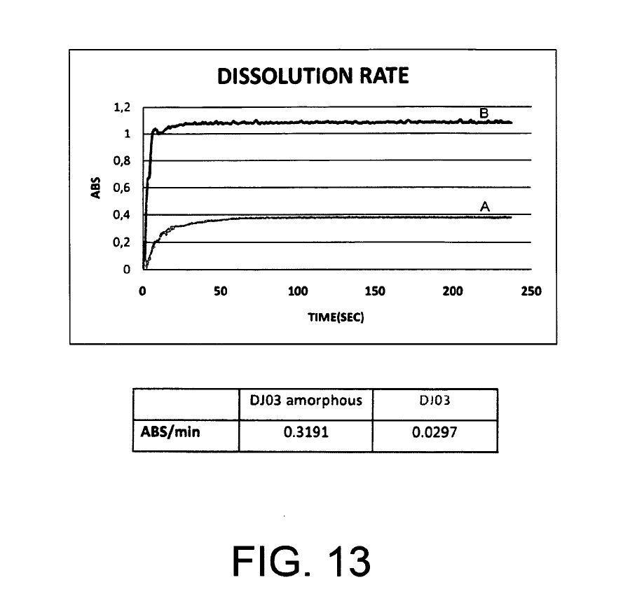

Dissolution tests were carried out on Aliskiren hemifumarate, amorphous solid, of this invention and Aliskiren hemifumarate, amorphous solid, obtained as described in WO2008/061622. Kinetic Tests were carried out using a Hanson Vision Classic 6 dissolution tester combined with a Varian Cary 50 UV-Vis spectrophotometer. The program used was “Kinetic” (Cary 50 WinUV software V.3) that continuously recorded absorbance at 280 nm of a buffer solution (80 ml) continuously stirred (100 rpm) at 37° C. to which the sample had been added. FIG. 13 shows the dissolution speed of Aliskiren hemifumarate, amorphous solid of this invention (curve A) and Aliskiren hemifumarate, amorphous solid obtained as described in WO2008/061622 (curve B).

### EXAMPLE 4

### Stability Test

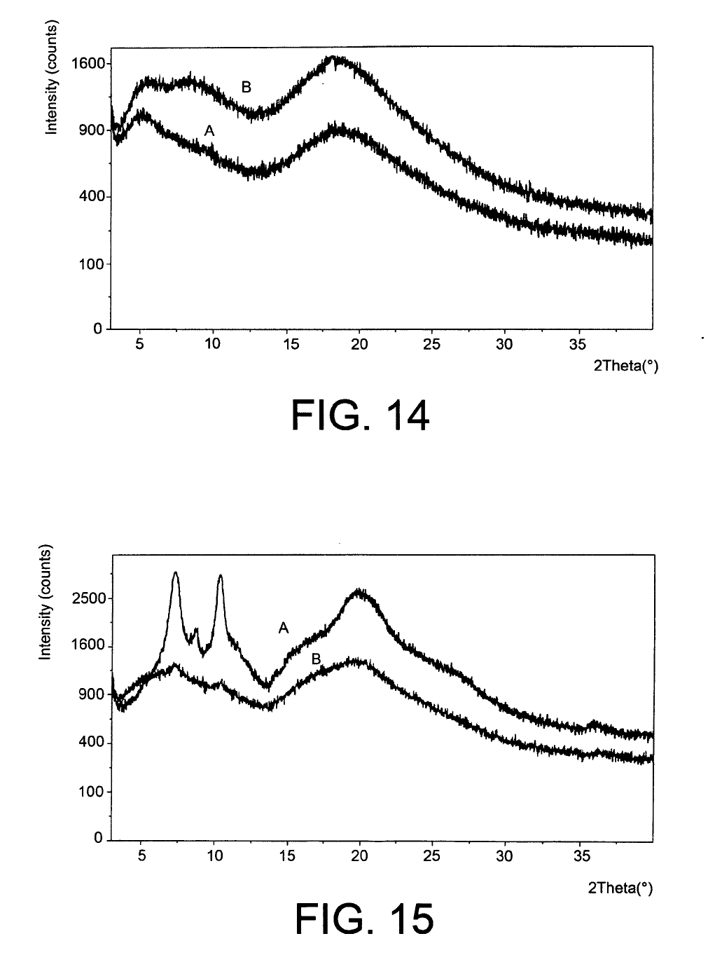

Stability tests were carried out on Aliskiren hemifumarate, amorphous solid of this invention and Aliskiren hemifumarate, amorphous solid obtained as described in WO2008/061622, maintaining the samples at 25° C., 75% RH. After 2 hours in said conditions, both samples changed from solid to viscous liquid form. The same samples were exposed to 25° C., 60% RH. In these conditions, the diffraction pattern assumed by Aliskiren hemifumarate, amorphous solid, obtained as described in WO2008/061622 is shown in FIG. 14, in which curve A is obtained in basal conditions, curve B is obtained after exposure of the sample for 5 days to the conditions indicated. FIG. 15 shows the diffraction pattern obtained exposing the Aliskiren hemifumarate, amorphous solid of this invention to the same conditions (curve A: basal; curve B: 5 days at 25° C., 60% RH). A comparison of curves A and B of FIG. 15 reveals that the typical nature of the diffractogram of Aliskiren hemifumarate, amorphous solid of this invention is lost exposing this for 5 days to the conditions indicated.

### EXAMPLE 5

### Compressibility Test

A set of tablets was prepared using different compression forces. A compressibility test was carried out on these. Table 7 shows the data obtained compressing Aliskiren hemifumarate, amorphous solid of this invention; Table 8 refers to Aliskiren hemifumarate, amorphous solid obtained as described in WO2008/061622.

TABLE 7

| Aliskiren | | | | | |
|---|---|---|---|---|---|
| hemifumarate | Compression | | | | Rupture |
| amorphous | strength | Diameter | Thickness | Weight | Strength |
| solid # | (kN) | (mm) | (mm) | (mg) | (N) |
| 1 | 11.83 | 7.02 | 1.34 | 54 | 4.5 |
| 2 | 5.83 | 7.01 | 1.18 | 48 | 4 |
| 3 | 16.45 | 7.03 | 1.2 | 50 | 2 |
| 4 | 10.85 | 7.00 | 1.13 | 45 | 4 |
| 5 | 19.43 | 7.00 | 1.29 | 52 | 3.5 |

TABLE 8

| Aliskiren | | | | | |
|---|---|---|---|---|---|
| hemifumarate | Compression | | | | Rupture |
| amorphous | strength | Diameter | Thickness | Weight | Strength |
| solid # | (kN) | (mm) | (mm) | (mg) | (N) |
| 1 | 22.27 | 7.01 | 1.16 | 46 | 1.5 |
| 2 | 23.82 | 7.03 | 1.19 | 50 | 2 |
| 3 | 24.15 | 7.03 | 1.22 | 50 | 1.5 |
| 4 | 6.80 | 6.99 | 1.12 | 44 | 2 |
| 5 | 7.95 | 7.04 | 1.12 | 46 | 2 |
| 6 | 10.10 | 7.02 | 1.18 | 48 | 1.5 |
| 7 | 11.55 | 7.01 | 1.22 | 48 | 2.2 |
| 8 | 7.88 | 7.02 | 1.16 | 45 | 2.5 |
| 9 | 10.15 | 7.00 | 1.12 | 48 | 2 |
| 10 | 13.18 | 7.01 | 1.23 | 54 | 2 |
| 11 | 15.60 | 7.04 | 1.26 | 53 | 2 |
| 12 | 14.12 | 7.06 | 1.25 | 50 | 2 |

A comparison of the data obtained demonstrated that Aliskiren hemifumarate, amorphous solid of this invention has a higher rupture strength than that observed in tablets of Aliskiren hemifumarate, amorphous solid obtained as described in WO2008/061622.

1. An amorphous form of Aliskiren hemifumarate with a melting point between 87 and 107° C.

2. The amorphous form of Aliskiren hemifumarate according to claim 1, wherein a XRPD diffractogram exhibits the following main peaks a 2theta+/−0.3 degrees: 7,3, 10.2, 10.4, 19.6.

3. The amorphous form according to claim 2, characterised by the following XRPD diffractogram of FIG. 9:

4. A process for preparing an amorphous form of Aliskiren hemifumarate ethyl acetate solvate that comprises:
i) dissolving Aliskiren hemifumarate in alcohol and heating to a suitable temperature;
ii) coating the solution obtained in i) to room temperature and spiking of same with pure Aliskiren hemifumarate;
iii) stirring the mix obtained in ii) at a suitable temperature and for a suitable time;
iv) further cooling the mix and continuing the stirring;
v) filtering the mix obtained in iv) in order to isolate the precipitate;
vi) washing the precipitate with alcohol and drying under vacuum.

5. The process according to claim 4, wherein said phase i) stirring is carried out at a temperature of between 35 and 55° C. and said alcohol is selected from the group consisting of methanol, ethanol, propanol, butanol, isopropanol, and isobutanol.

6. The process according to claim 4, wherein said phase iii) stirring is continued for 10-20 hours at a temperature of between about 20 and about 25° C. and in said phase iv) is cooled to a temperature below 10° C. and said stirring is continued for 2 hours or more and in said phase vi) the alcohol is selected in the group that comprises methanol, ethanol, propanol, butanol, isopropanol, and isobutanol, and said washing is repeated twice.

7. The process according to claim 4, in which said drying under vacuum takes place at a temperature below 50° C.

8. Pharmaceutical composition comprising as active ingredient the amorphous form of Aliskiren hemifumarate of claim 1.

9. A crystal form of Aliskiren hemifumarate that has a melting point of between 96.6 and 110° C.

10. The crystal form according to claim 9, wherein the XRPD diffractogram shows the following main peaks a 2theta+/−0.3 degrees: 5.8, 5.9, 9,6, 10.8, 15.6, 16.4, 18.4, 18.9.

11. The crystal form according to claim 10, characterised by the following XRPD diffractogram of FIG. 1:

12. The crystal form according to claim 9 that is obtained through desolvation of Aliskiren hemifumarate p-xylene solvate.

13. A process for the preparation of a crystal form of Aliskiren hemifumarate comprising:
i) re-suspending the Aliskiren hemifumarate in a suitable solvent, continuously stirred at room temperature in an oil bath;
ii) heating to a suitable temperature until a clear solution is obtained;
iii) cooling, continuing to stir, the solution obtained in ii) to room temperature;
iv) stirring the solution obtained in iii) at a suitable temperature and for a suitable time;
v) filtering the mix obtained in iv) in order to isolate the precipitate;
vi) drying the precipitate at a suitable temperature.

14. The process according to claim 13, wherein said phase ii) takes place at a temperature of between 60 and 90° C. and said solvent is selected in the group that comprises benzene, toluene, and xylene.

15. The process according to claim 13, wherein in said phase iii) said solution reaches room temperature in about 5 hours, in oil bath.

16. The process according to claim 13, wherein in said phase iv) said stirring is continued for a further 10 hours or more.

17. The process according to claim 13, wherein in said phase vi) said drying takes place at a temperature of between 50 and 90° C.

18. Pharmaceutical composition comprising as active ingredient the crystal form of Aliskiren hemifumarate according to claim 9.

19. An amorphous form of Aliskiren hemifumarate according to claim 1, wherein the melting point is at about 98.5° C.

20. The process according to claim 4, wherein said phase i) stirring is carried out at a temperature at about 40-45° C. and said alcohol is isopropanol.

21. The process according to claim 4, wherein said phase iii) stirring is continued for about 15 hours at a temperature of between about 20 and about 25° C. and in said phase iv) is cooled to a temperature between about 0 and about 5° C. and said stirring is continued for 2 hours or more and in said phase vi) the alcohol is cold isopropanol and said washing is repeated twice.

22. The process according to claim 4, wherein said drying under vacuum takes place at a temperature below about 40° C.

23. A crystal form of Aliskiren hemifumarate according to claim 9, wherein the melting point is about 105.4° C.

24. The process according to claim 13, in which said phase ii) takes place at a temperature at about 75° C., and said solvent is p-xylene.

25. The process according to claim 13, wherein in said phase iv) said stirring is continued for about 12 hours at room temperature.

26. The process according to claim 13, wherein in said phase vi) said drying takes place at a temperature at about 70° C. for about 3 hours.
